(19) United States
(12) Patent Application Publication
(10) Pub. No.: US 2025/0259111 A1
(43) Pub. Date: Aug. 14, 2025

(54) LEARNING APPARATUS, PHYSICAL PROPERTY PREDICTION APPARATUS, LEARNING PROGRAM, AND PHYSICAL PROPERTY PREDICTION PROGRAM

(71) Applicant: FUJIFILM CORPORATION, Tokyo (JP)

(72) Inventors: Takuya KITAMURA, Kanagawa (JP); Hiroshi ISHIKAWA, Kanagawa (JP)

(21) Appl. No.: 19/196,745

(22) Filed: May 2, 2025

Related U.S. Application Data
(63) Continuation of application No. PCT/JP2023/037928, filed on Oct. 19, 2023.

(30) Foreign Application Priority Data
Nov. 30, 2022 (JP) 2022-192326

(51) Int. Cl. G06N 20/00; G06F 30/27

(52) U.S. Cl. CPC G06N 20/00; G06F 30/27

(57) ABSTRACT

A learning apparatus executes: a similar structure data group selection step of selecting, based on first distance indicator for evaluating a degree of similarity in structure data between plural compounds, a similar structure data group that is a set of structure data of similar compounds similar to a reference compound, from a structure data group that is a set of structure data of plural compounds; a classification step of classifying, based on second distance indicator for evaluating the degree of similarity in the structure data between the plural compounds, the similar structure data group into selected group that is selected as training data for training a physical property prediction model and non-selected group; and a learning step of training the physical property prediction model that predicts physical properties of compounds of the non-selected group, using the selected group classified in the classification step as the training data.

### CROSS-REFERENCE TO RELATED APPLICATIONS

This application is a continuation of International Application No. PCT/JP2023/037928, filed on Oct. 19, 2023, which claims priority from Japanese Application No. 2022-192326, filed on Nov. 30, 2022. The entire disclosure of each of the above applications is incorporated herein by reference.

### BACKGROUND

### Technical Field

The present disclosure relates to a learning apparatus, a physical property prediction apparatus, a learning program, and a physical property prediction program.

### Related Art

In the related art, a physical property of a compound is predicted from structure data of the compound using a physical property prediction model obtained by machine learning. For example, in a case of developing a new material, the physical properties of a compound group in which a part or all of the structures are changed, with a specific compound group having known performance as a starting point, for performance improvement are predicted.

Therefore, there is a demand for a technology related to a learning apparatus for constructing a physical property prediction model that can predict the physical property of the compound from the structure data of the compound with high accuracy. For example, JP2021-76890A discloses a technology of using two compounds selected from a compound database as selected compounds and training a physical property prediction model using training data including at least a combination of a common structure and a differential structure of the selected compounds and the properties of the selected compounds.

In the technology disclosed in JP2021-76890A, in order to achieve high accuracy of the physical property prediction model, the training of the physical property prediction model may become inefficient. For example, in the technology disclosed in JP2021-76890A, in order to achieve high accuracy of the physical property prediction model, a relatively large amount of training data is required. Therefore, there is a demand for a technology capable of efficiently training the physical property prediction model that can predict the physical property with high accuracy.

### SUMMARY

The present disclosure provides a learning apparatus, a physical property prediction apparatus, a learning program, and a physical property prediction program with which a physical property prediction model that predicts a physical property of a compound from structure data of the compound with high accuracy can be efficiently trained.

A first aspect of the present disclosure relates to a learning apparatus that trains a physical property prediction model that is a learning model used in a physical property prediction apparatus that receives an input of structure data representing a structure of a compound to output a prediction result of a physical property of the compound, the learning apparatus comprising: a processor, in which the processor executes: a similar structure data group selection step of selecting, based on a first distance indicator for evaluating a degree of similarity in the structure data between a plurality of compounds, a similar structure data group that is a set of structure data of similar compounds similar to a reference compound, which is a reference, from a structure data group that is a set of structure data of a plurality of compounds; a classification step of classifying, based on a second distance indicator for evaluating the degree of similarity in the structure data between the plurality of compounds, the similar structure data group into a selected group that is selected as training data for training the physical property prediction model and a non-selected group that is other than the selected group; and a learning step of training the physical property prediction model that predicts physical properties of compounds of the non-selected group, using the selected group classified in the classification step as the training data.

In a second aspect of the present disclosure, according to the first aspect, the classification step may include a selected group evaluation step of evaluating a degree of similarity between groups of the selected group and the non-selected group based on an individual degree of similarity between individual pieces of the structure data included in each of the selected group and the non-selected group, the individual degree of similarity being derived based on the second distance indicator, and a determination step of determining whether or not an evaluation result of the selected group evaluation step satisfies a preset criterion.

In a third aspect of the present disclosure, according to the second aspect, the degree of similarity between the groups may be a degree of similarity between ratios of different types of structure data included in each of the selected group and the non-selected group.

In a fourth aspect of the present disclosure, according to the second aspect, the learning apparatus may further comprise: an addition step of adding a part of the non-selected group to the selected group in a case in which the evaluation result does not satisfy the preset criterion, and the processor may repeat the selected group evaluation step and the determination step based on the selected group and the non-selected group that are updated by the addition step, until the evaluation result satisfies the criterion.

In a fifth aspect of the present disclosure, according to the fourth aspect, in the addition step, a predetermined number of pieces of the structure data may be added from the structure data of the compounds included in the non-selected group to the selected group in descending order of the individual degree of similarity derived based on the second distance indicator.

In a sixth aspect of the present disclosure, according to the first aspect, the structure data group may be generated by using a derived data generation model using artificial intelligence, based on the structure data of the reference compound set in advance.

In a seventh aspect of the present disclosure, according to the first aspect, the first distance indicator may be any one of a Mahalanobis distance, cosine similarity, a Tanimoto coefficient, or a Euclidean distance between any descriptors representing compounds.

In an eighth aspect of the present disclosure, according to the first aspect, the first distance indicator may be a Tanimoto coefficient between any descriptors representing compounds.

In a ninth aspect of the present disclosure, according to the first aspect, the second distance indicator may be any one of a Mahalanobis distance, cosine similarity, a Tanimoto coefficient, or a Euclidean distance between any descriptors representing compounds.

In a tenth aspect of the present disclosure, according to the first aspect, the second distance indicator may be a Mahalanobis distance between any descriptors representing compounds.

In an eleventh aspect of the present disclosure, according to the first aspect, in the learning step, a trained physical property prediction model, which has been trained using compounds other than the selected group classified in the classification step as training data, may be trained using the selected group classified in the classification step as the training data.

In a twelfth aspect of the present disclosure, according to the first aspect, in the similar structure data group selection step, the similar structure data group may be selected based on the first distance indicator using a feature value represented by a fingerprint or a feature value output from an autoencoder as a descriptor of the structure data.

In a thirteenth aspect of the present disclosure, according to the first aspect, in the similar structure data group selection step, the similar structure data group may be selected based on the first distance indicator using a feature value represented by a Morgan fingerprint as a descriptor of the structure data.

A fourteenth aspect of the present disclosure relates to a physical property prediction apparatus that receives an input of structure data representing a structure of a compound to output a prediction result of a physical property of the compound using a physical property prediction model that is a learning model, the physical property prediction apparatus comprising: a processor, in which the processor executes: a similar structure data group selection step of selecting, based on a first distance indicator for evaluating a degree of similarity in the structure data between a plurality of compounds, a similar structure data group that is a set of structure data of similar compounds similar to a reference compound, which is a reference, from a structure data group that is a set of structure data of a plurality of compounds; a classification step of classifying, based on a second distance indicator for evaluating the degree of similarity in the structure data between the plurality of compounds, the similar structure data group into a selected group that is selected as training data for training the physical property prediction model and a non-selected group that is other than the selected group; a learning step of training the physical property prediction model using the selected group classified in the classification step as the training data; and a prediction step of inputting structure data representing a structure of a compound included in compounds of the non-selected group to the physical property prediction model trained in the learning step, to acquire a prediction result output from the physical property prediction model.

A fifteenth aspect of the present disclosure relates to a learning program causing a processor included in a learning apparatus that trains a physical property prediction model that is a learning model used in a physical property prediction apparatus that receives an input of structure data representing a structure of a compound to output a prediction result of a physical property of the compound, to execute: a similar structure data group selection step of selecting, based on a first distance indicator for evaluating a degree of similarity in the structure data between a plurality of compounds, a similar structure data group that is a set of structure data of similar compounds similar to a reference compound, which is a reference, from a structure data group that is a set of structure data of a plurality of compounds; a classification step of classifying, based on a second distance indicator for evaluating the degree of similarity in the structure data between the plurality of compounds, the similar structure data group into a selected group that is selected as training data for training the physical property prediction model and a non-selected group that is other than the selected group; and a learning step of training the physical property prediction model that predicts physical properties of compounds of the non-selected group, using the selected group classified in the classification step as the training data.

A sixteenth aspect of the present disclosure relates to a physical property prediction program causing a processor included in a physical property prediction apparatus that receives an input of structure data representing a structure of a compound to output a prediction result of a physical property of the compound using a physical property prediction model that is a learning model, to execute: a similar structure data group selection step of selecting, based on a first distance indicator for evaluating a degree of similarity in the structure data between a plurality of compounds, a similar structure data group that is a set of structure data of similar compounds similar to a reference compound, which is a reference, from a structure data group that is a set of structure data of a plurality of compounds; a classification step of classifying, based on a second distance indicator for evaluating the degree of similarity in the structure data between the plurality of compounds, the similar structure data group into a selected group that is selected as training data for training the physical property prediction model and a non-selected group that is other than the selected group; a learning step of training the physical property prediction model using the selected group classified in the classification step as the training data; and a prediction step of inputting structure data representing a structure of a compound included in compounds of the non-selected group to the physical property prediction model trained in the learning step, to acquire a prediction result output from the physical property prediction model.

According to the above-described aspects, with the learning apparatus, the physical property prediction apparatus, the learning program, and the physical property prediction program of the present disclosure, it is possible to efficiently train the physical property prediction model that predicts the physical property of the compound from the structure data of the compound with high accuracy.

### BRIEF DESCRIPTION OF THE DRAWINGS

FIG. 1 is a block diagram showing an example of a hardware configuration of a physical property prediction apparatus according to an exemplary embodiment.

FIG. 2 is a functional block diagram showing an example of a configuration related to a learning function of the physical property prediction apparatus according to the exemplary embodiment.

FIG. 3 is an explanatory diagram showing an example of a reference compound and a structure data group.

FIG. 4 is an explanatory diagram showing selection of a similar structure data group.

FIG. 5 is an explanatory diagram showing classification of a selected group and a non-selected group.

FIG. 6 is a diagram showing an example of a preset criterion used for determining an evaluation result.

FIG. 7 is a flowchart showing an example of learning processing executed by a processor according to the exemplary embodiment.

FIG. 8 is a functional block diagram showing an example of a configuration related to a prediction function of the physical property prediction apparatus according to the exemplary embodiment.

FIG. 9 is a flowchart showing an example of prediction processing executed by the processor according to the exemplary embodiment.

### DESCRIPTION

Hereinafter, exemplary embodiments for carrying out the technology of the present disclosure will be described in detail with reference to the drawings.

A physical property prediction apparatus according to the present exemplary embodiment has a function (hereinafter, referred to as a “learning function”) of training a physical property prediction model that is a learning model used in the physical property prediction apparatus that receives an input of structure data representing a structure of a compound to output a prediction result of a physical property of the compound. In addition, the physical property prediction apparatus according to the present exemplary embodiment has a function (hereinafter, referred to as a “prediction function”) of receiving an input of the structure data representing the structure of the compound and outputting the prediction result of the physical property of the compound by using the physical property prediction model that is a learning model.

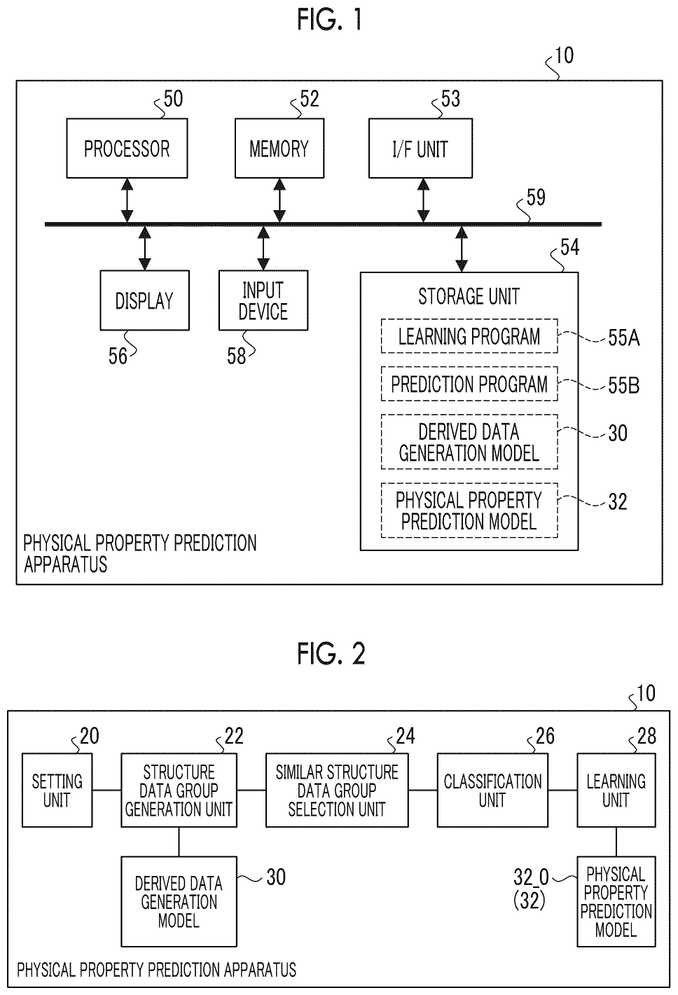

FIG. 1 is a block diagram showing an example of a hardware configuration of a physical property prediction apparatus 10 according to the present exemplary embodiment. As shown in FIG. 1, the physical property prediction apparatus 10 comprises a processor 50, such as a central processing unit (CPU), a memory 52, an interface (I/F) unit 53, a storage unit 54, a display 56, and an input device 58. The processor 50, the memory 52, the I/F unit 53, the storage unit 54, the display 56, and the input device 58 are connected to each other via a bus 59 such as a system bus or a control bus so that various types of information can be exchanged therebetween.

The processor 50 reads out various programs including a learning program 55A and a prediction program 55B stored in the storage unit 54 to the memory 52 and executes processing in accordance with the readout programs. As a result, the processor 50 controls the physical property prediction apparatus 10. The memory 52 is a work memory that is used in a case in which the processor 50 executes processing.

The learning program 55A and the prediction program 55B, which are executed in the processor 50, are stored in the storage unit 54. In addition, the storage unit 54 according to the present exemplary embodiment stores a derived data generation model 30 and a physical property prediction model 32, which will be described in detail later. Specific examples of the storage unit 54 include a hard disk drive (HDD) and a solid-state drive (SSD).

The I/F unit 53 communicates various types of information with an external device (not shown) or the like via wireless communication or wired communication. The display 56 and the input device 58 function as a user interface. The display 56 provides various types of information regarding the prediction result of the physical property of the compound to a user. The display 56 is not particularly limited, and examples of the display 56 include a liquid crystal monitor and a light emitting diode (LED) monitor. In addition, the input device 58 is operated by the user to input various instructions regarding the prediction of the physical property of the compound. The input device 58 is not particularly limited, and examples of the input device 58 include a keyboard, a touch pen, and a mouse. It should be noted that, in the physical property prediction apparatus 10, a touch panel display in which the display 56 and the input device 58 are integrated is adopted.

### Learning Function

First, the learning function in the physical property prediction apparatus 10 according to the present exemplary embodiment will be described. The configuration related to the learning function of the physical property prediction apparatus 10 according to the present exemplary embodiment is an example of a learning apparatus of the present disclosure.

FIG. 2 is a functional block diagram showing an example of the configuration related to the learning function of the physical property prediction apparatus 10 according to the present exemplary embodiment. As shown in FIG. 2, the physical property prediction apparatus 10 comprises a setting unit 20, a structure data group generation unit 22, a similar structure data group selection unit 24, a classification unit 26, and a learning unit 28. As an example, in the physical property prediction apparatus 10 according to the present exemplary embodiment, in a case in which the processor 50 executes the learning program 55A stored in the storage unit 54, the processor 50 functions as the setting unit 20, the structure data group generation unit 22, the similar structure data group selection unit 24, the classification unit 26, and the learning unit 28.

The setting unit 20 has a function of setting structure data of a reference compound that serves as a reference for a chemical structure for predicting the physical property.

In the physical property prediction apparatus 10 according to the present exemplary embodiment, the physical properties of a plurality of compounds in which a part or all of the structures are changed with the reference compound as a starting point are predicted. Therefore, the setting unit 20 sets the reference compound as a starting point. As an example, in the physical property prediction apparatus 10 according to the present exemplary embodiment, the user inputs the structure data of the reference compound using the input device 58. The setting unit 20 acquires the structure data input by the user using the input device 58 and sets the acquired structure data as the structure data of the reference compound.

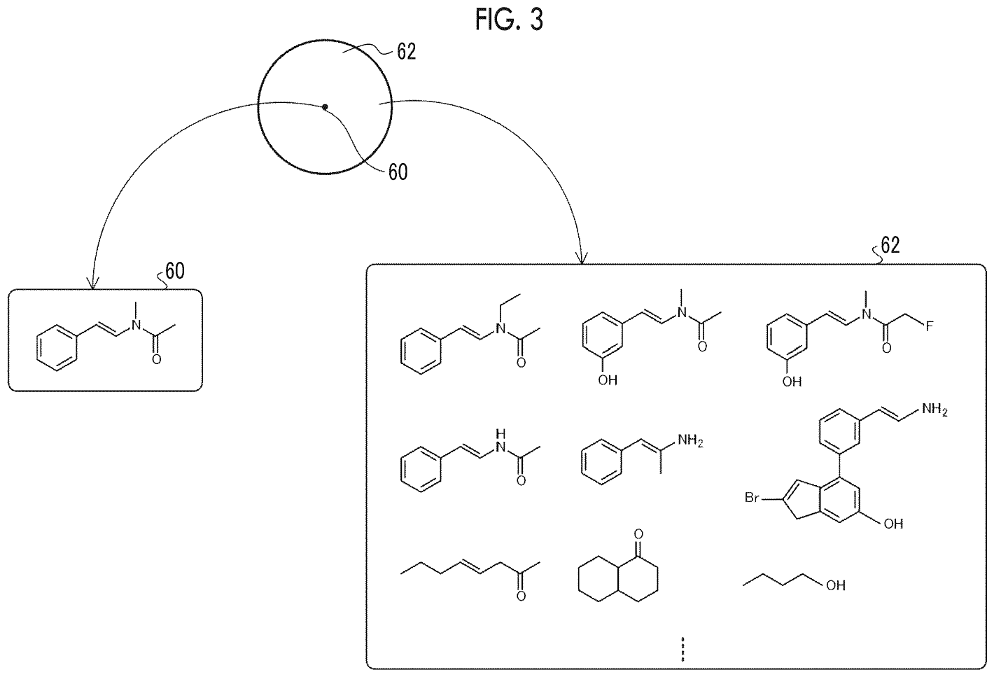

The structure data group generation unit 22 has a function of generating structure data of a plurality of peripheral compounds in which a part or all of the structures of the reference compound are changed, based on the structure data of the set reference compound. Here, the structure data of the reference compound and the structure data of the plurality of peripheral compounds will be referred to as a “structure data group”. FIG. 3 shows an example of a reference compound 60 and a structure data group 62. It should be noted that, in FIG. 3, as a specific example of the structure data group 62, the structure data of nine compounds are shown, but the number of pieces of the structure data of the compounds included in the structure data group 62 is not limited to nine. The number of pieces of the structure data of the compounds included in the structure data group 62 may be determined in consideration of, for example, the number of compounds used as training data, which will be described later, the accuracy of the physical property prediction model 32 to be constructed, and the processing load related to learning processing.

As an example, the structure data group generation unit 22 according to the present exemplary embodiment generates the structure data of the plurality of peripheral compounds by using the derived data generation model 30 using artificial intelligence. Examples of the derived data generation model 30 include a Variational AutoEncoder (VAE), a reinforcement learning model, a genetic algorithm, and a generative adversarial networks (GAN) model. As described above, it is possible to easily generate a large amount of the structure data of the peripheral compounds by using the derived data generation model 30.

The structure data group generation unit 22 outputs the generated structure data group 62 to the similar structure data group selection unit 24.

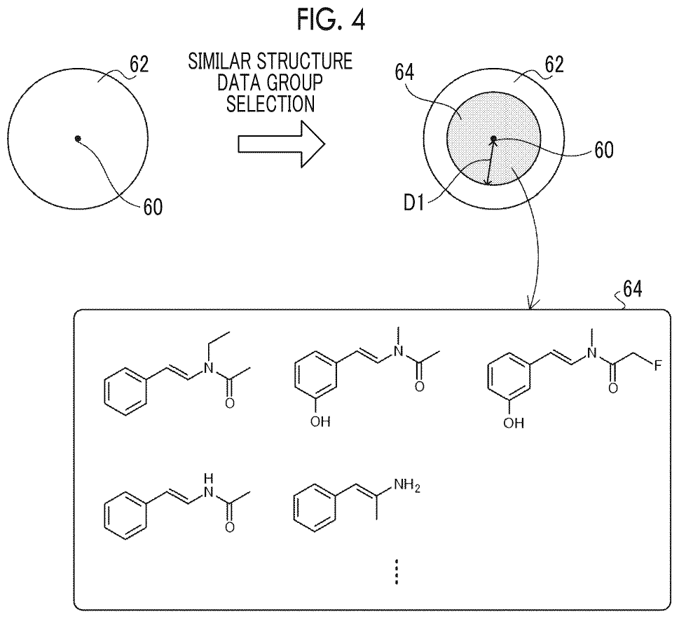

As shown in FIG. 4, the similar structure data group selection unit 24 has a function of selecting a similar structure data group 64, which is a set of structure data of similar compounds similar to the reference compound 60, from the structure data group 62 based on a first distance indicator (distance D1). FIG. 4 shows an example of the similar structure data group 64 selected by the similar structure data group selection unit 24 from the structure data group 62 shown in FIG. 3. It should be noted that the number of pieces of the structure data of the similar compounds included in the similar structure data group 64 is not limited to the number of pieces (five) of the structure data of the similar compounds shown in FIG. 4.

The first distance indicator is an indicator for evaluating a degree of similarity in the structure data between the plurality of compounds. Examples of the first distance indicator include a Mahalanobis distance, a cosine similarity, a Tanimoto coefficient (Tanimoto distance), and a Euclidean distance between any descriptors representing compounds. It is preferable to use the Tanimoto coefficient (Tanimoto distance) as the first distance indicator. The Tanimoto coefficient is an indicator obtained by converting a molecular structure of the compound into a fingerprint and calculating the degree of similarity between the fingerprints. Specifically, as a Tanimoto coefficient between a compound A and a compound B, a Tanimoto coefficient is used, which is a value obtained by dividing the number of partial structures common to the compound A and the compound B by the total number of partial structures included in the compound A and the compound B. The closer the Tanimoto coefficient is to 1, the higher the degree of similarity between compounds, and the closer the Tanimoto coefficient is to 0, the lower the degree of similarity between compounds. It should be noted that, instead of the Tanimoto coefficient, the Tanimoto distance may be used, and the Tanimoto distance is a value obtained by subtracting the Tanimoto coefficient from 1. Therefore, the closer the Tanimoto distance is to 0, the higher the degree of similarity between the compounds, and the closer the Tanimoto distance is to 1, the lower the degree of similarity between the compounds.

For example, in a case in which the first distance indicator is the Tanimoto distance, the similar structure data group selection unit 24 selects the structure data of a compound of which the distance DI from the reference compound 60 is less than a predetermined distance (<1), as the structure data of the similar compound. It should be noted that the predetermined distance, which is a threshold value for determining that the compound is similar to the reference compound 60, varies depending on the type of the first distance indicator, but a specific value thereof is not particularly limited and may be determined, for example, depending on the number of pieces of the structure data of the compounds that are the similar structure data group 64.

In addition, the descriptor representing the compound is an indicator that determines the physical property of the compound, and represents a feature value related to the physical property with a numerical value. Examples of the descriptor include a feature value represented by a fingerprint and a feature value output from an autoencoder. It should be noted that, among these examples, it is preferable to use, as the descriptor, a feature value represented by a Morgan fingerprint that expresses the structure of the compound by the number of partial structures at a certain distance from an atom.

The similar structure data group selection unit 24 outputs the selected similar structure data group 64 to the classification unit 26.

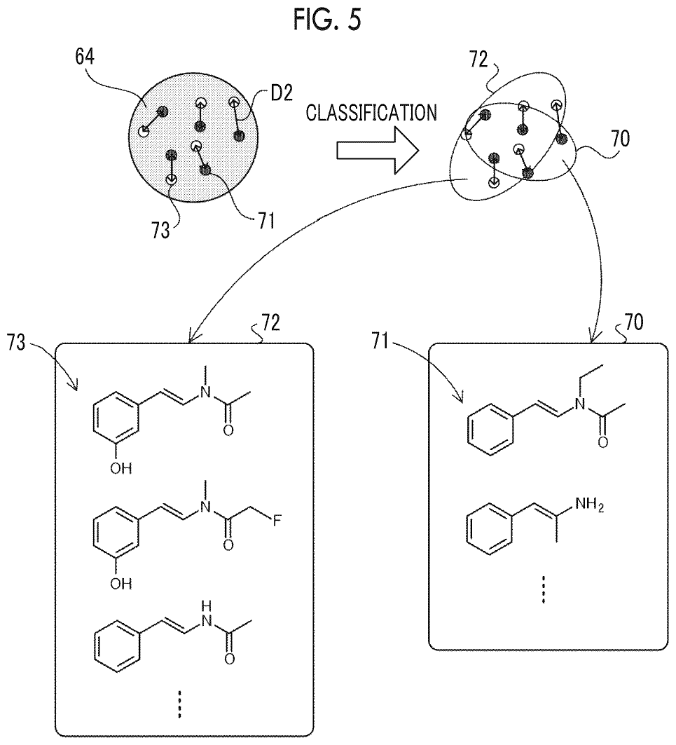

As shown in FIG. 5, the classification unit 26 has a function of classifying the similar structure data group 64 into a selected group 70 selected as the training data used for training the physical property prediction model 32 and a non-selected group 72 other than the selected group 70, based on a second distance indicator (distance D2). FIG. 5 shows an examples of the selected group 70 selected from the similar structure data group 64 shown in FIG. 4 and the non-selected group 72. It should be noted that the number of pieces of the structure data 71 of the compounds included in the selected group 70 and the number of pieces of the structure data 73 of the compounds included in the non-selected group 72 are not limited to the number of pieces (two) of the structure data 71 and the number of pieces (three) of the structure data 73 shown in FIG. 5.

The second distance indicator is an indicator for evaluating the degree of similarity in the structure data between the plurality of compounds. Examples of the second distance indicator include a Mahalanobis distance, a cosine similarity, a Tanimoto coefficient (Tanimoto distance), and a Euclidean distance between any descriptors representing compounds. The type of the first distance indicator and the type of the second distance indicator may be different from each other or may be the same as each other, but it is preferable that the Mahalanobis distance is used as the second distance indicator. The Mahalanobis distance is a distance in consideration of a correlation between the compounds, and the smaller the Mahalanobis distance, the more similar the compounds are.

Specifically, the classification unit 26 according to the present exemplary embodiment derives an individual degree of similarity between the structure data 71 of the compound included in the selected group 70 and the structure data 73 of the compound included in the non-selected group 72, based on the second distance indicator. For example, the classification unit 26 derives the Mahalanobis distance (distance D2) between the structure data 71 of the compound included in the selected group 70 and the structure data 73 of the compound included in the non-selected group 72, as the degree of similarity. It should be noted that, in the present exemplary embodiment, in order to avoid complicated description, the distance D2 between the structure data 71 of the compound included in the selected group 70 and the structure data 73 of the compound included in the non-selected group 72 may be referred to as the “distance D2 between the selected group 70 and the non-selected group 72”.

In addition, the classification unit 26 evaluates the degree of similarity between the groups of the selected group 70 and the non-selected group 72 based on the individual degree of similarity of the structure data of the compound, and determines whether or not an evaluation result satisfies a preset criterion. That is, the classification unit 26 according to the present exemplary embodiment derives a distance between the descriptors, for example, the Mahalanobis distance as the individual degree of similarity, and determines whether or not the evaluation result based on the Mahalanobis distance satisfies the preset criterion.

The degree of similarity between the groups in the present exemplary embodiment is a degree of similarity of ratios of different types of the structure data included in each of the selected group 70 and the non-selected group 72. That is, the classification unit 26 determines whether or not the evaluation result of the degree of similarity between the ratios of the different types of the structure data included in each of the selected group 70 and the non-selected group 72 satisfies the preset criterion.

Examples of the preset criterion include a criterion using a specific numerical value of the distance indicator in a case in which the evaluation result is a value of the distance indicator. For example, a value of the distance indicator of “20” may be set as the preset criterion. In this case, the classification unit 26 determines that the criterion is satisfied in a case in which the distance D2 between the selected group 70 and the non-selected group 72 is equal to or less than “20”.

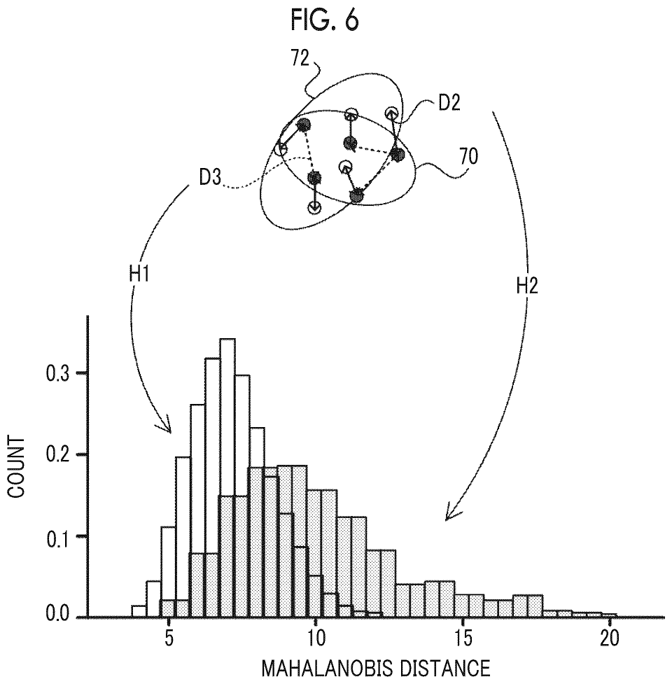

In addition, examples of the preset criterion include, as shown in FIG. 6, a criterion determined based on a comparison result between the distance D2 between the selected group 70 and the non-selected group 72 and a distance D3 between the structure data 71 of the compounds included in the selected group 70. In the present exemplary embodiment, in order to avoid complication of description, the distance D3 between the structure data 71 of the compounds included in the selected group 70 may be referred to as the “distance D3 between the selected groups 70”.

In a case of using this criterion, for example, the classification unit 26 need only divide the number of pieces of the structure data 71 of the compounds included in the selected group 70 into two structure data groups and derive the distance D3 between the structure data 71 included in one structure data group and the structure data 71 included in the other structure data group.

In a case of using this criterion, a ratio of a histogram H2 representing a distribution of the distance D2 between the selected group 70 and the non-selected group 72 to a histogram H3 representing a distribution of the distance D3 between the selected groups 70 is used as the evaluation result. The higher the ratio of the histogram H2 included in the histogram H3, the higher the degree of similarity between the groups of the selected group 70 and the non-selected group 72. Therefore, in a case in which the preset criterion is set to, for example, “equal to or higher than 80%”, the classification unit 26 determines that the criterion is satisfied in a case in which the ratio of the histogram H2 included in the histogram H3 is “equal to or higher than 80%”. It should be noted that, in the example shown in FIG. 6, since the ratio of the histogram H2 included in the histogram H3 is 40%, the classification unit 26 determines that the preset criterion is not satisfied.

In addition, in a case in which the evaluation result does not satisfy the preset criterion, the classification unit 26 adds a part of the non-selected group 72 to the selected group 70. That is, the fact that the evaluation result does not satisfy the preset criterion means a state in which the ratios of the different types of the structure data included in the selected group 70 and the non-selected group 72 are not similar to each other. Therefore, the classification unit 26 adds a part of the non-selected group 72 to the selected group 70 until the evaluation result satisfies the criterion. It is preferable that the structure data 73 to be added to the selected group 70 from the non-selected group 72 has a large individual degree of similarity (distance D2) derived based on the second distance indicator. For example, a predetermined number of pieces of the structure data 73 are added to the selected group 70 in descending order of the distance D2 among the structure data 73 included in the non-selected group 72. The structure data 73 included in the non-selected group 72 is added to the selected group 70 to become the structure data 71. The learning unit 28 repeats the evaluation of the degree of similarity between the groups and the determination of the evaluation result based on the selected group 70 and the non-selected group 72 that are updated by the addition of the structure data, until the preset criterion is satisfied.

The classification unit 26 outputs the selected group 70 out of the selected group 70 and the non-selected group 72 classified in this manner to the learning unit 28.

The learning unit 28 has a function of training the physical property prediction model 32 using the selected group 70 as the training data. Examples of the physical property prediction model 32 include a graph neural network (GNN) model that incorporates a deep learning mechanism into graph data.

It should be noted that the learning unit 28 may construct the physical property prediction model 32 using the selected group 70 as the training data for a general-purpose physical property prediction model that predicts the physical property of the compound from the structure data of the compound. For example, the learning unit 28 may construct the physical property prediction model 32 using the selected group 70 as the training data based on the physical property prediction model that has been trained in advance to predict the physical property using, as the training data, the structure data of a plurality of compounds randomly selected from The PubChem QC Project, which is a database of the structure data of the compounds. In this case, it is preferable to use the structure data of the compound other than the compounds included in the selected group 70 for the training of the original physical property prediction model.

The learning unit 28 stores the trained physical property prediction model 32 constructed in this manner in the storage unit 54.

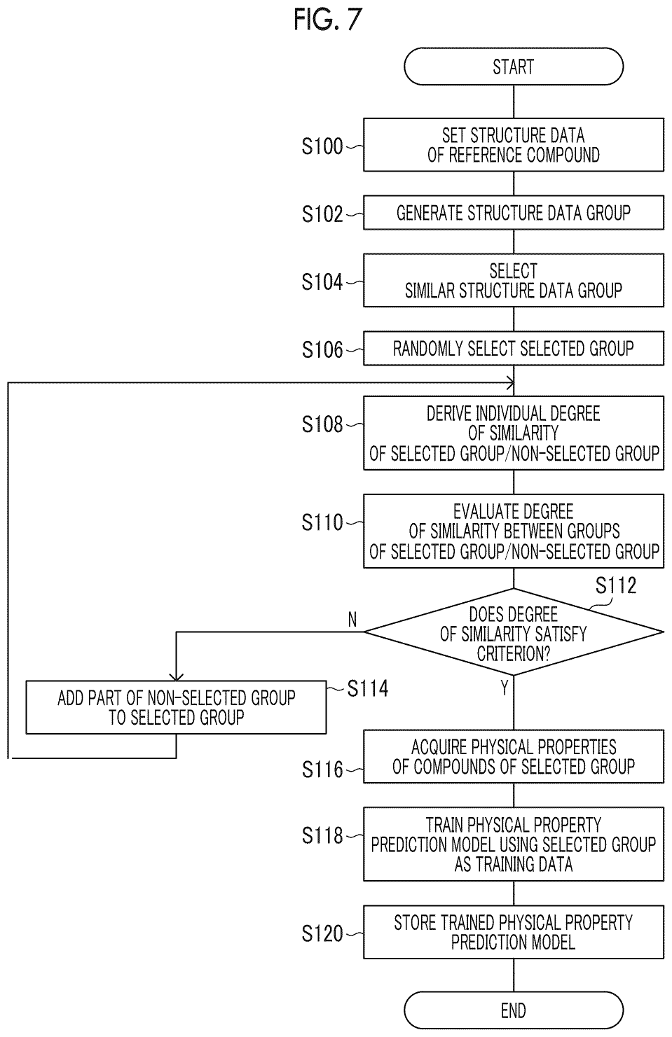

Next, an operation of the learning function of the physical property prediction apparatus 10 according to the present exemplary embodiment will be described. FIG. 7 is a flowchart showing an example of a flow of the learning processing executed by the processor 50 of the physical property prediction apparatus 10 according to the present exemplary embodiment. As an example, in the processor 50 according to the present exemplary embodiment, in a case in which an instruction to start the learning is received, the learning processing shown in FIG. 7 as an example is executed.

In step S100 of FIG. 7, as described above, the setting unit 20 sets the structure data input by the user as the structure data of the reference compound 60.

In next step S102, as described above, the structure data group generation unit 22 generates the structure data group 62 using the derived data generation model 30.

In next step S104, as described above, the similar structure data group selection unit 24 selects the similar structure data group 64 from the structure data group 62 based on the first distance indicator (distance D1). The processing of step S104 according to the present exemplary embodiment is an example of a similar structure data group selection step of the present disclosure.

In next step S106, the classification unit 26 randomly selects a predetermined number of compounds from the compounds included in the similar structure data group 64, to set the selected group 70. The number of compounds that are randomly selected in this step is an initial value of the number of selected groups 70. The number of compounds that are randomly selected by the classification unit 26 is, for example, about 1/10 to 1/20 of the number of compounds included in the similar structure data group 64. Specifically, in a case in which the number of compounds included in the similar structure data group 64 is 1,000, the classification unit 26 randomly selects 50 to 100 compounds.

It should be noted that, in a case in which the compounds included in the randomly selected compound group, that is, the compounds included in the initial selected group 70 are different, a final trained physical property prediction model 32 is strictly different. However, by repeating the processing of steps S108 to S114, which will be described later, a homogeneous physical property prediction model 32 is finally constructed.

In next step S108, as described above, the classification unit 26 derives the individual degree of similarity between the compound included in the selected group 70 and the compound included in the non-selected group 72 based on the second distance indicator (distance D2).

In next step S110, as described above, the classification unit 26 evaluates the degree of similarity between the groups of the selected group 70 and the non-selected group 72 based on the individual degree of similarity.

In next step S112, as described above, the classification unit 26 determines whether or not the degree of similarity satisfies the preset criterion. In a case in which the degree of similarity does not satisfy the criterion, a negative determination is made in the determination in step S112, and the processing proceeds to step S114.

In step S114, the classification unit 26 adds a part of the non-selected group 72 to the selected group 70. For example, as described above, the classification unit 26 adds the predetermined number of compounds to the selected group 70 from the non-selected group 72 in descending order of the individual degree of similarity based on the second distance indicator (distance D2) derived in step S108. In a case in which the processing of step S114 ends, the processing returns to step S108, and the processing of steps S108 and S110 is repeated.

On the other hand, in a case in which the degree of similarity satisfies the criterion, in step S112, an affirmative determination is made, and the processing proceeds to step S116. The processing of steps S106 to S114 according to the present exemplary embodiment is an example of a classification step of the present disclosure. It should be noted that the processing of step S110 according to the present exemplary embodiment is an example of a selected group evaluation step of the present disclosure, the processing of step S112 according to the present exemplary embodiment is an example of a determination step of the present disclosure, and the processing of step S114 according to the present exemplary embodiment is an example of an addition step of the present disclosure.

In step S116, the learning unit 28 acquires the physical property of each of the plurality of compounds included in the selected group 70. As an example, in the present exemplary embodiment, the user specifies the physical property of each of the plurality of compounds included in the selected group 70 by an experiment, calculation, or the like, and inputs the specified physical property by the input device 58. The learning unit 28 acquires the physical property input by the user.

In next step S118, the learning unit 28 trains the physical property prediction model 32 using the selected group 70 as the training data. Specifically, the physical property prediction model 32 is trained using a combination of the structure data of the compound included in the selected group 70 and the physical property of the compound, as the training data. It should be noted that step S116 and step S118 according to the present exemplary embodiment are an example of a learning step of the present disclosure.

In next step S120, the learning unit 28 stores the trained physical property prediction model 32 in the storage unit 54. In a case in which the processing of step S120 ends, the learning processing shown in FIG. 7 ends.

### Prediction Function

Next, the prediction function in the physical property prediction apparatus 10 according to the present exemplary embodiment will be described. The configuration related to the prediction function of the physical property prediction apparatus 10 according to the present exemplary embodiment is an example of a physical property prediction apparatus of the present disclosure.

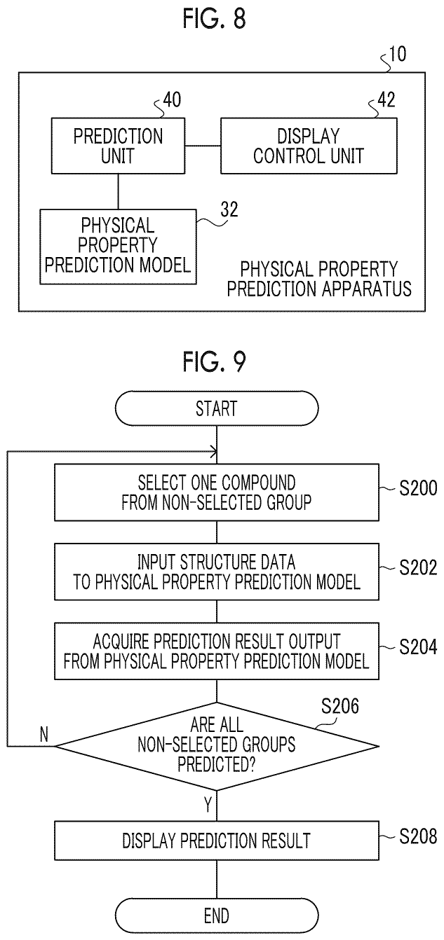

FIG. 8 is a functional block diagram showing an example of the configuration related to the prediction function of the physical property prediction apparatus 10 according to the present exemplary embodiment. As shown in FIG. 8, the physical property prediction apparatus 10 comprises a prediction unit 40 and a display control unit 42. As an example, in the physical property prediction apparatus 10 according to the present exemplary embodiment, in a case in which the processor 50 executes the prediction program 55B stored in the storage unit 54, the processor 50 functions as the prediction unit 40 and the display control unit 42.

The prediction unit 40 has a function of inputting the structure data representing the structure of the compound included in the non-selected group 72 to the physical property prediction model 32 trained by the learning processing (see FIG. 7) described above, to acquire a prediction result output from the physical property prediction model 32. The prediction unit 40 outputs the prediction result to the display control unit 42.

The display control unit 42 has a function of displaying the prediction result, that is, the prediction result of the physical property of each compound included in the non-selected group 72, on the display 56. It should be noted that the prediction result is not limited to being displayed, and may be stored in the storage unit 54 or output to the external device via the I/F unit 53.

Next, an operation of the prediction function of the physical property prediction apparatus 10 according to the present exemplary embodiment will be described. FIG. 9 is a flowchart showing an example of a flow of prediction processing executed by the processor 50 of the physical property prediction apparatus 10 according to the present exemplary embodiment. The processor 50 executes the prediction processing shown in FIG. 9, for example, after the end of the learning processing (see FIG. 7) or in a case in which an instruction to start the prediction is received.

In step S200 of FIG. 9, the prediction unit 40 selects one compound from the non-selected group 72.

In next step S202, the prediction unit 40 inputs the structure data of the selected compound to the physical property prediction model 32. Then, in next step S204, the prediction unit 40 acquires the prediction result output from the physical property prediction model 32.

In next step S206, the prediction unit 40 determines whether or not the physical properties of all the compounds included in the non-selected group 72 are predicted. In a case in which there is a compound for which the physical property is not predicted, a negative determination is made in step S206, the processing returns to step S200, and the processing of steps S200 to S204 is repeated. On the other hand, in a case in which the physical properties of all the compounds included in the non-selected group 72 are predicted, an affirmative determination is made in step S206, and the processing proceeds to step S208. It should be noted that the processing of steps S200 to S206 in the present exemplary embodiment is an example of a prediction step of the present disclosure.

In step S208, as described above, the display control unit 42 displays the prediction result on the display 56. In a case in which the processing of step S208 ends, the prediction processing shown in FIG. 9 ends.

As described above, in the physical property prediction apparatus 10 according to the present exemplary embodiment, first, the physical property prediction model 32 is constructed through machine learning. The similar structure data group selection unit 24 selects, from the structure data group 62 which is a set of the structure data of the plurality of compounds, the similar structure data group 64 which is a set of the structure data of the similar compounds similar to the reference compound 60, based on the first distance indicator (distance D1) for evaluating the degree of similarity in the structure data between the plurality of compounds. The classification unit 26 classifies the compounds included in the similar structure data group 64 into the selected group 70 and the non-selected group 72 such that the degree of similarity between the compounds included in the selected group 70 and the compounds included in the non-selected group 72 based on the second distance indicator (distance D2) satisfies the preset criterion. Further, the physical property prediction apparatus 10 trains the physical property prediction model 32 for predicting the non-selected group 72 using the selected group 70 classified by the classification unit 26, as the training data.

In this way, the classification unit 26 classifies the similar structure data group 64 into the selected group 70 that is the training data and the non-selected group 72 that is the prediction target, so that the selected group 70 and the non-selected group 72 include the structure data of different types at approximately the same ratio. Since the degree of similarity between the training data and the prediction target is high, it is possible to construct the physical property prediction model 32 that can predict the physical property of the compound from the structure data of the compound of the prediction target with high accuracy. In addition, with the physical property prediction apparatus 10 according to the present exemplary embodiment, it is possible to predict the physical property of the compound having a wide range of features.

In addition, in the physical property prediction apparatus 10 according to the present exemplary embodiment, as described above, the prediction unit 40 predicts the physical properties of the compounds in the non-selected group 72 by the physical property prediction model 32 trained using the selected group 70 as the training data.

Therefore, with the physical property prediction apparatus 10 according to the present exemplary embodiment, the physical property prediction model 32 that predicts the physical property of the compound from the structure data of the compound with high accuracy can be efficiently trained. In addition, with the physical property prediction model 32 trained as described above, the physical property of the compound can be predicted with high accuracy. It should be noted that, in the present exemplary embodiment, the form has been described in which the structure data group 62 is the peripheral compound of the reference compound 60, but the structure data group 62 need not be the peripheral compound of the reference compound 60 and may be, for example, any compound. Further, the structure data group 62 may be generated without using the derived data generation model 30. For example, the user or the like may manually generate the compound included in the structure data group 62. In this case, combinatorial synthesis and the like may be used.

It should be noted that, in the learning processing (see FIG. 7) described above, a case has been described in which the degree of similarity between the groups satisfies the preset criterion at least finally, but in a case in which the preset criterion is strict, there is a case in which the criterion is not satisfied or a case in which the criterion is not easily satisfied. In such a case, for example, the preset criterion may be relaxed. For example, in the learning processing shown in FIG. 7, in a case in which the preset criterion is not satisfied even though the processing of step S114 of adding a part of the non-selected group 72 to the selected group 70 is performed a predetermined number of times, the preset criterion may be changed to a criterion that is more relaxed than the current criterion, and then the processing of step S106 may be performed again. It should be noted that, in a case in which the preset criterion is relaxed, there is a concern that the prediction accuracy of the physical property prediction model 32 to be finally constructed is lowered. Since the preset criterion and the prediction accuracy of the physical property prediction model 32 are in a trade-off relationship, it is preferable to determine a degree of the preset criterion in consideration of the prediction accuracy of the physical property prediction model 32.

In addition, in the present exemplary embodiment, only a case has been described in which a part of the non-selected group 72 is added to the selected group 70, but the degree of similarity between the groups of the selected group 70 and the non-selected group 72 that are updated by adding a part of the non-selected group 72 to the selected group 70 may be low. In such a case, a part of the non-selected group 72 added to the selected group 70 may be returned to the non-selected group 72 again, and another compound may be added from the non-selected group 72 to the selected group 70.

In addition, in the present exemplary embodiment, the form has been described in which the physical property prediction apparatus 10 has both the learning function and the prediction function, and is an example of the learning apparatus and the physical property prediction apparatus of the present disclosure, but the present disclosure is not limited to this form. For example, the learning apparatus that performs the learning function and the physical property prediction apparatus that performs the prediction function may be provided as different apparatuses.

In the present exemplary embodiment, for example, as a hardware structure of a processing unit that executes various types of processing, such as the setting unit 20, the structure data group generation unit 22, the similar structure data group selection unit 24, the classification unit 26, the learning unit 28, the prediction unit 40, and the display control unit 42, various processors shown below can be used. As described above, in addition to the CPU that is a general-purpose processor that executes software (program) to function as various processing units, the various processors include a programmable logic device (PLD) that is a processor whose circuit configuration can be changed after manufacture, such as a field programmable gate array (FPGA), and a dedicated electric circuit that is a processor having a circuit configuration that is designed for exclusive use in order to execute specific processing, such as an application specific integrated circuit (ASIC).

One processing unit may be configured by one of the various processors or may be configured by a combination of two or more processors of the same type or different types (for example, a combination of a plurality of FPGAs or a combination of a CPU and an FPGA). A plurality of processing units may be configured by one processor.

A first example of the configuration in which the plurality of processing units are configured by one processor is a form in which one processor is configured by a combination of one or more CPUs and the software and this processor functions as the plurality of processing units, as represented by computers such as a client and a server. A second example is a form of using a processor that implements the function of the entire system including the plurality of processing units via one integrated circuit (IC) chip, as represented by a system on a chip (SoC) or the like. As described above, as the hardware structure, the various processing units are configured by using one or more of the various processors described above.

Further, the hardware structure of the various processors is, more specifically, an electric circuit (circuitry) in which circuit elements, such as semiconductor elements, are combined.

In addition, in each of the above-described exemplary embodiments, the aspect has been described in which each of the learning program 55A and the prediction program 55B is stored (installed) in the storage unit 54 in advance, but the present disclosure is not limited to this. The learning program 55A and the prediction program 55B may be provided in a form of being recorded in a recording medium, such as a compact disc read only memory (CD-ROM), a digital versatile disc read only memory (DVD-ROM), and a universal serial bus (USB) memory. In addition, each of the learning program 55A and the prediction program 55B may be downloaded from the external device via a network. That is, the program (program product) described in the present exemplary embodiment may be distributed from an external computer, in addition to being provided using the recording medium.

The following supplementary notes will be further disclosed in regard to the above-described exemplary embodiments.

### Supplementary Note 1

A learning apparatus that trains a physical property prediction model that is a learning model used in a physical property prediction apparatus that receives an input of structure data representing a structure of a compound to output a prediction result of a physical property of the compound, the learning apparatus comprising: a processor, in which the processor executes: a similar structure data group selection step of selecting, based on a first distance indicator for evaluating a degree of similarity in the structure data between a plurality of compounds, a similar structure data group that is a set of structure data of similar compounds similar to a reference compound, which is a reference, from a structure data group that is a set of structure data of a plurality of compounds; a classification step of classifying, based on a second distance indicator for evaluating the degree of similarity in the structure data between the plurality of compounds, the similar structure data group into a selected group that is selected as training data for training the physical property prediction model and a non-selected group that is other than the selected group; and a learning step of training the physical property prediction model that predicts physical properties of compounds of the non-selected group, using the selected group classified in the classification step as the training data.

### Supplementary Note 2

The learning apparatus according to supplementary note 1, in which the classification step includes a selected group evaluation step of evaluating a degree of similarity between groups of the selected group and the non-selected group based on an individual degree of similarity between individual pieces of the structure data included in each of the selected group and the non-selected group, the individual degree of similarity being derived based on the second distance indicator, and a determination step of determining whether or not an evaluation result of the selected group evaluation step satisfies a preset criterion.

### Supplementary Note 3

The learning apparatus according to supplementary note 2, in which the degree of similarity between the groups is a degree of similarity between ratios of different types of structure data included in each of the selected group and the non-selected group.

### Supplementary Note 4

The learning apparatus according to supplementary note 2, further comprising: an addition step of adding a part of the non-selected group to the selected group in a case in which the evaluation result does not satisfy the preset criterion, in which the processor repeats the selected group evaluation step and the determination step based on the selected group and the non-selected group that are updated by the addition step, until the evaluation result satisfies the criterion.

### Supplementary Note 5

The learning apparatus according to supplementary note 4, in which, in the addition step, a predetermined number of pieces of the structure data are added from the structure data of the compounds included in the non-selected group to the selected group in descending order of the individual degree of similarity derived based on the second distance indicator.

### Supplementary Note 6

The learning apparatus according to any one of supplementary notes 1 to 5, in which the structure data group is generated by using a derived data generation model using artificial intelligence, based on the structure data of the reference compound set in advance.

### Supplementary Note 7

The learning apparatus according to any one of supplementary notes 1 to 6, in which the first distance indicator is any one of a Mahalanobis distance, cosine similarity, a Tanimoto coefficient, or a Euclidean distance between any descriptors representing compounds.

### Supplementary Note 8

The learning apparatus according to any one of supplementary notes 1 to 6, in which the first distance indicator is a Tanimoto coefficient between any descriptors representing compounds.

### Supplementary Note 9

The learning apparatus according to any one of supplementary notes 1 to 8, in which the second distance indicator is any one of a Mahalanobis distance, cosine similarity, a Tanimoto coefficient, or a Euclidean distance between any descriptors representing compounds.

### Supplementary Note 10

The learning apparatus according to any one of supplementary notes 1 to 8, in which the second distance indicator is a Mahalanobis distance between any descriptors representing compounds.

### Supplementary Note 11

The learning apparatus according to any one of supplementary notes 1 to 10, in which, in the learning step, a trained physical property prediction model, which has been trained using compounds other than the selected group classified in the classification step as training data, is trained using the selected group classified in the classification step as the training data.

### Supplementary Note 12

The learning apparatus according to any one of supplementary notes 1 to 11, in which, in the similar structure data group selection step, the similar structure data group is selected based on the first distance indicator using a feature value represented by a fingerprint or a feature value output from an autoencoder as a descriptor of the structure data.

### Supplementary Note 13

The learning apparatus according to any one of supplementary notes 1 to 11, in which, in the similar structure data group selection step, the similar structure data group is selected based on the first distance indicator using a feature value represented by a Morgan fingerprint as a descriptor of the structure data.

### Supplementary Note 14

A physical property prediction apparatus that receives an input of structure data representing a structure of a compound to output a prediction result of a physical property of the compound using a physical property prediction model that is a learning model, the physical property prediction apparatus comprising: a processor, in which the processor executes: a similar structure data group selection step of selecting, based on a first distance indicator for evaluating a degree of similarity in the structure data between a plurality of compounds, a similar structure data group that is a set of structure data of similar compounds similar to a reference compound, which is a reference, from a structure data group that is a set of structure data of a plurality of compounds; a classification step of classifying, based on a second distance indicator for evaluating the degree of similarity in the structure data between the plurality of compounds, the similar structure data group into a selected group that is selected as training data for training the physical property prediction model and a non-selected group that is other than the selected group; a learning step of training the physical property prediction model using the selected group classified in the classification step as the training data; and a prediction step of inputting structure data representing a structure of a compound included in compounds of the non-selected group to the physical property prediction model trained in the learning step, to acquire a prediction result output from the physical property prediction model.

### Supplementary Note 15

A learning program causing a processor included in a learning apparatus that trains a physical property prediction model that is a learning model used in a physical property prediction apparatus that receives an input of structure data representing a structure of a compound to output a prediction result of a physical property of the compound, to execute: a similar structure data group selection step of selecting, based on a first distance indicator for evaluating a degree of similarity in the structure data between a plurality of compounds, a similar structure data group that is a set of structure data of similar compounds similar to a reference compound, which is a reference, from a structure data group that is a set of structure data of a plurality of compounds; a classification step of classifying, based on a second distance indicator for evaluating the degree of similarity in the structure data between the plurality of compounds, the similar structure data group into a selected group that is selected as training data for training the physical property prediction model and a non-selected group that is other than the selected group; and a learning step of training the physical property prediction model that predicts physical properties of compounds of the non-selected group, using the selected group classified in the classification step as the training data.

### Supplementary Note 16

A physical property prediction program causing a processor included in a physical property prediction apparatus that receives an input of structure data representing a structure of a compound to output a prediction result of a physical property of the compound using a physical property prediction model that is a learning model, to execute: a similar structure data group selection step of selecting, based on a first distance indicator for evaluating a degree of similarity in the structure data between a plurality of compounds, a similar structure data group that is a set of structure data of similar compounds similar to a reference compound, which is a reference, from a structure data group that is a set of structure data of a plurality of compounds; a classification step of classifying, based on a second distance indicator for evaluating the degree of similarity in the structure data between the plurality of compounds, the similar structure data group into a selected group that is selected as training data for training the physical property prediction model and a non-selected group that is other than the selected group; a learning step of training the physical property prediction model using the selected group classified in the classification step as the training data; and a prediction step of inputting structure data representing a structure of a compound included in compounds of the non-selected group to the physical property prediction model trained in the learning step, to acquire a prediction result output from the physical property prediction model.

The entire disclosure of Japanese Patent Application No. 2022-192326 filed on Nov. 30, 2022 is incorporated into the present specification by reference.

All of the documents, the patent applications, and the technical standards described in the present specification are incorporated in the present specification by reference to the same extent as in a case in which each of the documents, the patent applications, and the technical standards are specifically and individually described to be incorporated by reference.

What is claimed is:

1. A learning apparatus that trains a physical property prediction model that is a learning model used in a physical property prediction apparatus that receives an input of structure data representing a structure of a compound to output a prediction result of a physical property of the compound, the learning apparatus comprising:
a processor, wherein the processor executes:
a similar structure data group selection step of selecting, based on a first distance indicator for evaluating a degree of similarity in the structure data between a plurality of compounds, a similar structure data group that is a set of structure data of similar compounds similar to a reference compound, which is a reference, from a structure data group that is a set of structure data of a plurality of compounds;
a classification step of classifying, based on a second distance indicator for evaluating the degree of similarity in the structure data between the plurality of compounds, the similar structure data group into a selected group that is selected as training data for training the physical property prediction model and a non-selected group that is other than the selected group; and
a learning step of training the physical property prediction model that predicts physical properties of compounds of the non-selected group, using the selected group classified in the classification step as the training data.

2. The learning apparatus according to claim 1, wherein the classification step includes
a selected group evaluation step of evaluating a degree of similarity between groups of the selected group and the non-selected group based on an individual degree of similarity between individual pieces of the structure data included in each of the selected group and the non-selected group, the individual degree of similarity being derived based on the second distance indicator, and
a determination step of determining whether or not an evaluation result of the selected group evaluation step satisfies a preset criterion.

3. The learning apparatus according to claim 2, wherein the degree of similarity between the groups is a degree of similarity between ratios of different types of structure data included in each of the selected group and the non-selected group.

4. The learning apparatus according to claim 2, further comprising an addition step of adding a part of the non-selected group to the selected group, in a case in which the evaluation result does not satisfy the preset criterion,
wherein the processor repeats the selected group evaluation step and the determination step based on the selected group and the non-selected group that are updated by the addition step, until the evaluation result satisfies the criterion.

5. The learning apparatus according to claim 4, wherein, in the addition step, a predetermined number of pieces of the structure data are added from the structure data of the compounds included in the non-selected group to the selected group in descending order of the individual degree of similarity derived based on the second distance indicator.

6. The learning apparatus according to claim 1, wherein the structure data group is generated by using a derived data generation model using artificial intelligence, based on the structure data of the reference compound set in advance.

7. The learning apparatus according to claim 1, wherein the first distance indicator is any one of a Mahalanobis distance, cosine similarity, a Tanimoto coefficient, or a Euclidean distance between any descriptors representing compounds.

8. The learning apparatus according to claim 1, wherein the first distance indicator is a Tanimoto coefficient between any descriptors representing compounds.

9. The learning apparatus according to claim 1, wherein the second distance indicator is any one of a Mahalanobis distance, cosine similarity, a Tanimoto coefficient, or a Euclidean distance between any descriptors representing compounds.

10. The learning apparatus according to claim 1, wherein the second distance indicator is a Mahalanobis distance between any descriptors representing compounds.

11. The learning apparatus according to claim 1, wherein, in the learning step, a trained physical property prediction model, which has been trained using compounds other than the selected group classified in the classification step as training data, is trained using the selected group classified in the classification step as the training data.

12. The learning apparatus according to claim 1, wherein, in the similar structure data group selection step, the similar structure data group is selected based on the first distance indicator using a feature value represented by a fingerprint or a feature value output from an autoencoder as a descriptor of the structure data.

13. The learning apparatus according to claim 1, wherein, in the similar structure data group selection step, the similar structure data group is selected based on the first distance indicator using a feature value represented by a Morgan fingerprint as a descriptor of the structure data.

14. A physical property prediction apparatus that receives an input of structure data representing a structure of a compound to output a prediction result of a physical property of the compound using a physical property prediction model that is a learning model, the physical property prediction apparatus comprising:
a processor, wherein the processor executes:
a similar structure data group selection step of selecting, based on a first distance indicator for evaluating a degree of similarity in the structure data between a plurality of compounds, a similar structure data group that is a set of structure data of similar compounds similar to a reference compound, which is a reference, from a structure data group that is a set of structure data of a plurality of compounds;
a classification step of classifying, based on a second distance indicator for evaluating the degree of similarity in the structure data between the plurality of compounds, the similar structure data group into a selected group that is selected as training data for training the physical property prediction model and a non-selected group that is other than the selected group;
a learning step of training the physical property prediction model using the selected group classified in the classification step as the training data; and
a prediction step of inputting structure data representing a structure of a compound included in compounds of the non-selected group to the physical property prediction model trained in the learning step, to acquire a prediction result output from the physical property prediction model.

15. A non-transitory computer readable medium storing a learning program causing a processor included in a learning apparatus that trains a physical property prediction model that is a learning model used in a physical property prediction apparatus that receives an input of structure data representing a structure of a compound to output a prediction result of a physical property of the compound, to execute:
a similar structure data group selection step of selecting, based on a first distance indicator for evaluating a degree of similarity in the structure data between a plurality of compounds, a similar structure data group that is a set of structure data of similar compounds similar to a reference compound, which is a reference, from a structure data group that is a set of structure data of a plurality of compounds;
a classification step of classifying, based on a second distance indicator for evaluating the degree of similarity in the structure data between the plurality of compounds, the similar structure data group into a selected group that is selected as training data for training the physical property prediction model and a non-selected group that is other than the selected group; and
a learning step of training the physical property prediction model that predicts physical properties of compounds of the non-selected group, using the selected group classified in the classification step as the training data.

16. A non-transitory computer readable medium storing a physical property prediction program causing a processor included in a physical property prediction apparatus that receives an input of structure data representing a structure of a compound to output a prediction result of a physical property of the compound using a physical property prediction model that is a learning model, to execute:
a similar structure data group selection step of selecting, based on a first distance indicator for evaluating a degree of similarity in the structure data between a plurality of compounds, a similar structure data group that is a set of structure data of similar compounds similar to a reference compound, which is a reference, from a structure data group that is a set of structure data of a plurality of compounds;
a classification step of classifying, based on a second distance indicator for evaluating the degree of similarity in the structure data between the plurality of compounds, the similar structure data group into a selected group that is selected as training data for training the physical property prediction model and a non-selected group that is other than the selected group;
a learning step of training the physical property prediction model using the selected group classified in the classification step as the training data; and
a prediction step of inputting structure data representing a structure of a compound included in compounds of the non-selected group to the physical property prediction model trained in the learning step, to acquire a prediction result output from the physical property prediction model.
